(19) United States
(12) Patent Application Publication
(10) Pub. No.: US 2021/0290940 A1
(43) Pub. Date: Sep. 23, 2021

(54) LVAD FIXATION AND INFECTION MANAGEMENT

(71) Applicant: Medtronic, Inc., Minneapolis, MN (US)

(72) Inventors: Kevin R. SEIFERT, Forest Lake, MN (US); Satish PULAPURA, Bridgewater, NJ (US); Melissa G. T. CHRISTIE, Ham Lake, MN (US); Rebecca L. TEMPLE, Chicago, IL (US); Michael D. EGGEN, Chisago City, MN (US)

(21) Appl. No.: 17/177,519

(22) Filed: Feb. 17, 2021

Related U.S. Application Data
(60) Provisional application No. 62/991,735, filed on Mar. 19, 2020.

(51) Int. Cl. A61M 60/861; A61M 60/17; A61M 60/873

(52) U.S. Cl. CPC A61M 60/861; A61M 60/17; A61M 2210/125; A61M 2205/0205; A61M 2205/0238; A61M 60/873

(57) ABSTRACT

A medical implant fixation device including a non-bioresorbable material sized and configured to one from the group consisting of receive and surround the medical implant and be affixed to a surface of the medical implant. A plurality of bioresorbable fixation projections extend from the non-bioresorbable material and at least one from the group consisting of the plurality of bioresorbable fixation projections and the non-bioresorbable material are coated with at least one anti-infection drug.

### CROSS-REFERENCE TO RELATED APPLICATION

This application claims the benefit of U.S. Application Ser. No. 62/991,735, filed Mar. 19, 2020.

### FIELD

The present technology is generally related to fixation devices for medical implants.

### BACKGROUND

A mechanical circulatory support device (MCSD) such as a left ventricular assist device (LVAD) is an implantable device that is used to assist the functioning of a failing heart. LVADs include a pump that connects the left ventricle to the aorta which pulls blood from the left ventricle and pumps it into the aorta. The pump is connected by a percutaneous driveline with an electrical wire to an external battery pack, which provides power to the pump. LVADs have evolved from the first generation which used volume-displacement pumps through axial flow pumps, to the latest continuous flow centrifugal or axial flow pumps. Infection rates associated with LVADs, however, are high. A recent review from the Mayo Clinic reported that the infection rate for first-generation LVADs vary from 25 to 80%, and for second generation 30 to 50%. It has been determined that sepsis (infection) caused twice as many deaths as device failure. Infections also increase the cost to the healthcare system. Moreover, with the advent of fully implantable systems having implantable blood pumps, such as LVADs, more electronic equipment is implanted within the body and within or proximate various types of tissue.

In particular, transcutaneous energy transfer (TET) systems are used to supply power MCSDs implanted within a human body. An electromagnetic field generated by a transmitting coil outside the body can transmit power across a cutaneous (skin) barrier to a magnetic receiving coil implanted within the body. The receiving coil can then transfer the received power to the implanted heart pump or other internal device and to one or more batteries implanted within the body. One of the challenges with TET systems are the material properties of the receiving coil and the resultant side effects on the patient. In particular, owing to the induction properties of the receiving coil, proper alignment with an external transmission coil is important such that the requisite power is transferred. Movement of the receiving coil can cause efficiency losses, causing excessive heating of surrounding tissue and patient discomfort, whereby the implantable coil stability plays an important role in minimizing heat generation and maintaining a high energy transfer efficiency.

### SUMMARY

The techniques of this disclosure generally relate to fixation devices for medical implants.

In one aspect, the present disclosure provides a medical implant fixation device including a non-bioresorbable material sized and configured to one from the group consisting of receive and surround the medical implant and be affixed to a surface of the medical implant. A plurality of bioresorbable fixation projections extend from the non-bioresorbable material and at least one from the group consisting of the plurality of bioresorbable fixation projections and the non-bioresorbable material are coated with at least one anti-infection drug.

In another aspect, the medical implant is a controller for a ventricular assist device, and wherein the non-bioresorbable material further defines a pouch sized to receive and enclose the controller.

In another aspect, the medical implant is a driveline for a ventricular assist device, and wherein the non-bioresorbable material defines a mesh is configured to be wrapped around and adhered to the driveline.

In another aspect, the driveline includes an elongated tubular body having at least one electrical connector configured to electrically connect with at least a portion of the ventricular assist device, and wherein the driveline further includes a biocompatible fabric wrapped around at least a portion of the elongated tubular body configured to promote tissue ingrowth, and wherein the non-bioresorbable material is wrapped around the biocompatible fabric.

In another aspect, the driveline includes a plurality of anchoring sleeves disposed about the driveline, and wherein non-bioresorbable material is disposed between two of the plurality of anchoring sleeves, and wherein the anchoring sleeves are configured to retain the non-bioresorbable material about the driveline.

In another aspect, the non-bioresorbable material defines a first side and a second side opposite the first side, and wherein the plurality of bioresorbable fixation projections is disposed on both the first side and the second side.

In another aspect, the medical implant is a transcutaneous energy coil.

In another aspect, the non-bioresorbable material is affixed around a perimeter of the transcutaneous energy coil.

In another aspect, the non-bioresorbable material is configured to be substantially planar with an outer surface of the transcutaneous energy coil.

In another aspect, the plurality of bioresorbable fixation projections define one from the group consisting of a plurality of barbs and a plurality of hooks.

In one aspect, a method of implanting a medical implant within a patient includes one from the group consisting of enclosing the medical implant within a fixation device and affixing the fixation device to the medical implant. The fixation device including a non-bioresorbable material. A plurality of bioresorbable fixation projections extend from the non-bioresorbable material and at least one from the group consisting of the plurality of bioresorbable fixation projections and the non-bioresorbable material are coated with at least one anti-infection drug. The medical implant and the fixation device are implanted within the patient.

In another aspect, the medical implant is a controller for a ventricular assist device, and wherein the controller is enclosed within the fixation device.

In another aspect, the medical implant is a driveline for a ventricular assist device, and wherein the fixation device is affixed to at least a portion of the driveline.

In another aspect, the fixation device is wrapped around at least portion of the driveline.

In another aspect, the driveline includes an elongated tubular body having at least one electrical connector configured to electrically connect with at least a portion of the ventricular assist device, and wherein the driveline further includes a biocompatible fabric wrapped around at least a portion of the elongated tubular body configured to promote tissue ingrowth, and wherein the non-bioresorbable material is wrapped around the biocompatible fabric.

In another aspect, the non-bioresorbable material defines a first side and a second side opposite the first side, and wherein the plurality of bioresorbable fixation projections is disposed on both the first side and the second side.

In another aspect, the medical implant is a transcutaneous energy coil.

In another aspect, the non-bioresorbable material is affixed around a perimeter of the transcutaneous energy coil.

In another aspect, the non-bioresorbable material is one from the group consisting of sewn, glue, and welded onto the transcutaneous energy coil.

In one aspect, a medical implant fixation device includes a non-bioresorbable material sized and configured to receive and enclose a controller for a ventricular assist device, an entirety of the non-bioresorbable material defining a mesh having a surface area. A plurality of bioresorbable fixation projections extend from the non-bioresorbable material, the plurality of bioresorbable fixation projections are disposed on substantially the entirety of the surface area, the plurality of bioresorbable fixation projections are configured to engage and anchor to subcutaneous adipose tissue. The plurality of bioresorbable fixation projections are coated with at least one anti-bacterial drug.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside of the body view of a patient with a ventricular assist device, receiving coil, and controller, fully implanted within the patient;

FIG. 2 is a front inside of the body view of a patient with a ventricular assist device percutaneously implanted within the patient;

FIG. 3 is a perspective view of a fixation device of the present application being disposed around a biocompatible fabric of a driveline;

FIG. 4 is a zoomed in view of the fixation device shown in FIG. 3;

FIG. 5 is another embodiment of the fixation device shown in FIG. 3 having anchors;

FIG. 6 is another embodiment of the fixation device shown in FIG. 3 defining an enclosure and surrounding a controller of a ventricular assist device;

FIG. 7 is another embodiment of the fixation device shown in FIG. 3 being wound about a portion of a TETS coil;

FIG. 8A is embodiment of the fixation device shown in FIG. 4 with an anti-infection mesh;

FIG. 8B is at top view of the anti-infection mesh disposed on the fixation device;

FIG. 8C is a top view of the anti-infection mesh and fixation device spiral wrapped about the driveline;

FIG. 9A is a perspective view of a fixation device of the present application in the form of a tube being disposed around a driveline in a first configuration; and

FIG. 9B is a perspective view of a fixation device of the present application in the form of a tube being disposed around a driveline in a second configuration.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

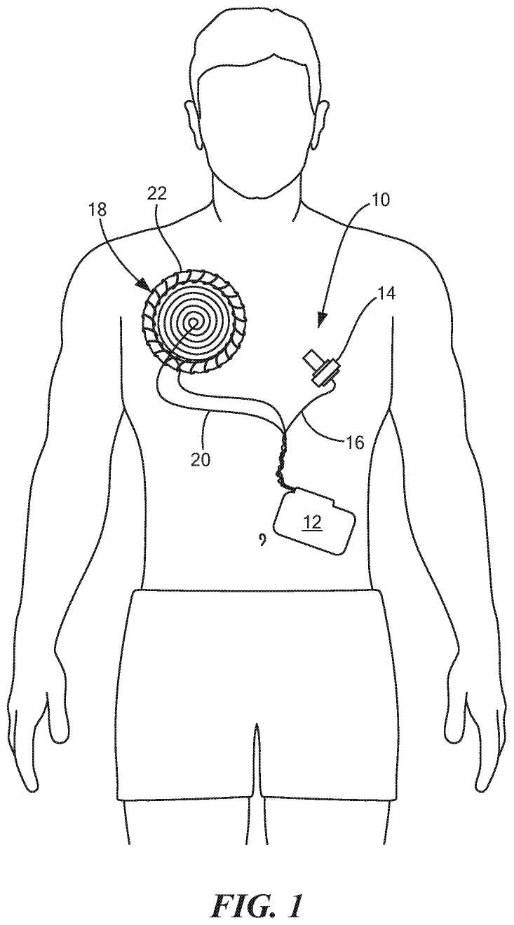

Referring now to the drawings in which like reference designators refer to like elements there is shown in FIG. 1 a portion of an exemplary transcutaneous energy transfer (TET) system constructed in accordance with the principles of the present application and designated generally as “10.” The system 10 is fully implantable within a patient, whether human or animal, which is to say there are no percutaneous connections between implanted components of the system 10 and components outside of the body of the patient. In the configuration shown in FIG. 1, the system 10 includes a controller 12 implanted within the body of the patient. The controller 12 may include a battery (not shown) configured to power the components of the controller and provide power one or more implantable medical device, for example, a ventricular assist device (VAD) 14 implanted within the left ventricle of the patient's heart. VADs 14 may include centrifugal pumps, axial pumps, or other kinds electromagnetic pumps configured to pump blood from the heart to blood vessels to circulate around the body. One such centrifugal pump is the HVAD sold by HeartWare, Inc. and is shown and described in U.S. Pat. No. 7,997,854 the entirety of which is incorporated by reference. One such axial pump is the MVAD sold by HeartWare, Inc. and is shown and described in U.S. Pat. No. 8,419,609 the entirety of which is incorporated herein by reference. In an exemplary configuration, the VAD 14 is electrically coupled to the controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving coil 18 may also be coupled to the controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission coil (not shown) coupled to an external battery (not shown) disposed opposite the receiving coil 18 on the outside of the patient's body. The receiving coil 18 may be disposed within a hermetically sealed package 22 that does not interfere with the conductivity of the receiving coil 18.

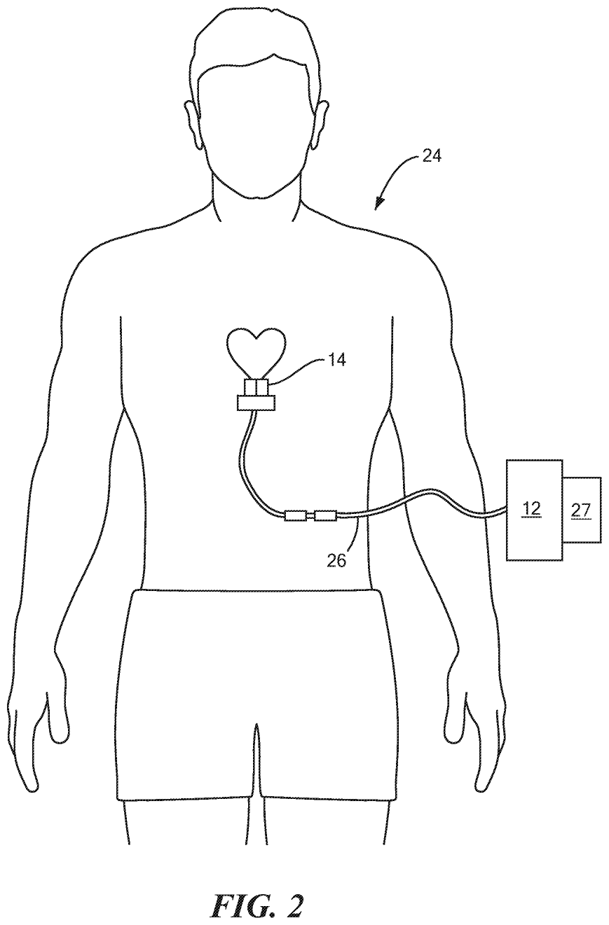

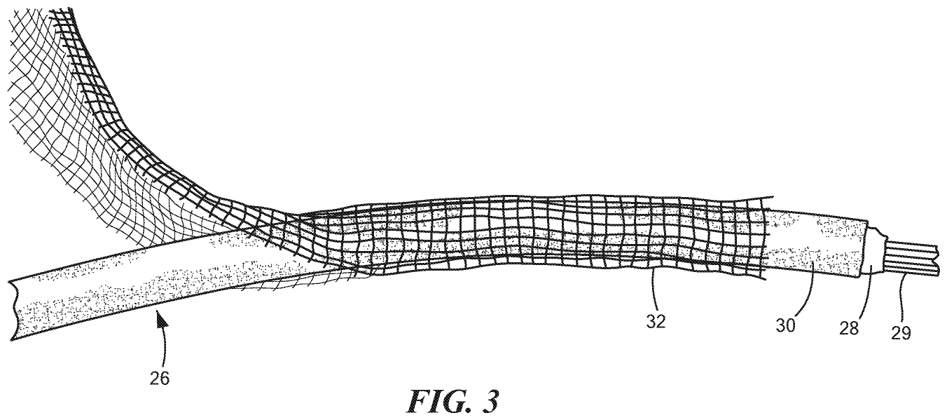

Referring now to FIG. 2, in an exemplary percutaneous system 24 constructed in accordance with the principles of the present application, the VAD 14 may be the same as the VAD 14 shown in FIG. 1, however, the VAD 14 receives power through a driveline 26 implanted within the body percutaneously connected to the controller 12, which is external to the body and coupled to a battery 27. The driveline 26 may include one or more conductors 29 (shown in FIG. 3) disposed within an elongated tubular body 28 that spans the distance between the VAD 14 and a percutaneous exit location on the abdomen. At least a portion of the elongated tubular body 28 may include a biocompatible fabric 30, for example Velour, circumferentially disposed about the elongated tubular body 28 to promote tissue ingrowth.

Referring now to FIG. 3, in either the TET system 10 shown in FIG. 1, or the percutaneous system 24 shown in FIG. 2, the risk of infection and/or undesirable movement of the implanted components may be minimized with the use of a medical implant fixation device 32. The fixation device 32 may include of a first material 34, which may be, for example, PRO-GRIP™ or V-Loc™ manufactured by Applicant, and configured to at least partially surround, enclose, or be affixed to the desired implanted component. In an exemplary configuration, the first material 34 is non-bioresorbable and defines a mesh to promote tissue ingrowth. In another configuration, the first material 34 may be bioresorbable. The first material 34 is a biocompatible material that is flexible as to accommodate differently sized medical implants. In the configuration shown in FIG. 3, the fixation device 32 including the first material 34 is at least partially wrapped around and adhered to at least a portion of the driveline 26, and in particular, the biocompatible fabric 30. In other configurations, the fixation device 32 is completely or partially wrapped around the tubular body 28 without the biocompatible fabric 30. The first material 34 may be wrapped around a portion of the length of the tubular body 28 or fabric 30 or substantially the entire length.

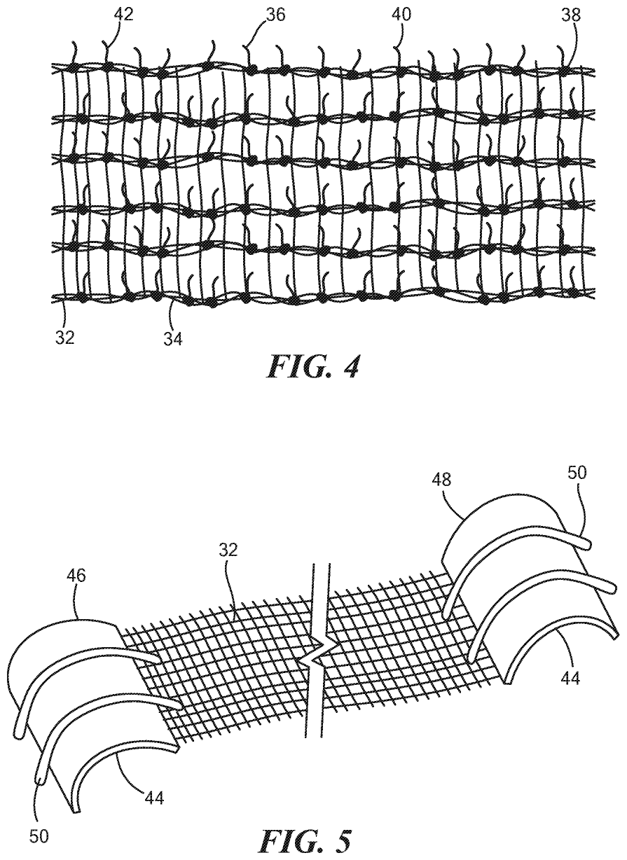

Referring now to FIG. 4, extending from the first material 34 is a plurality of fixation projections 36 configured to engage with tissue proximate the implant component of system 10, for example, adipose issue. In one configuration the plurality of fixations projections 36 are bioresorbable after a predetermined amount of time. In other configurations, plurality of fixations projections 36 are non-bioresorbable. The plurality of fixations projections 36 may be elongated in shape, for example, rod shaped, and may have a proximal end 38 coupled to the first material 34 and a distal end 40 that is a free end. For example, as shown in FIG. 4, the plurality of fixations projections 36 may define a junction with a mesh network of the first material 34 and extend outward away from the mesh. In one configuration, plurality of fixations projections 36 extend substantially orthogonally away from the first material 34. The length, thickness, and number of the plurality of fixations projections 36 may vary depending on the location in which the implanted component is implanted and the size of the implanted component. For example, in one configuration, the number of the plurality of fixations projections 36 may be increased when the first material 34 is coupled to or disposed around the controller 12, which is larger and heavier than compared to a driveline 26. The plurality of fixations projections 36 and/or the first material 34 may be the same or different materials depending on the desired properties. For example, the first material 34 and plurality of fixations projections 36 may both be non-bioresorbable, bioresorbable, or a one or the other.

The plurality of fixations projections 36 and/or the first material 34 may be coated or impregnated with one or more anti-infection drugs or coatings 42. For example, the plurality of fixations projections 36 may be coated with an antibiotic drug or a silver ion coating to prevent infection, such that antibiotic or silver ion is released upon implantation of the plurality of fixations projections 36. In other configurations, a reservoir (not shown) may be formed within one or more of the plurality of fixations projections 36, and within the reservoir the antibiotic or silver ion is retained. As the plurality of fixations projections 36 are resorbed into by the body, the antibiotic may be released as a bolus, or metered as a function of the degradation of anti-infection drug 42 on the surface of the plurality of projections 36 or the first material 34. For example, the time during which the anti-infection drug 42 is released may depend on the surface area of the plurality of fixations projections 36 coated with the anti-infection drug 42. Thus, if a longer or shorter period of time is desired for release of the anti-infection drug 42, the surface area of the plurality of fixations projections 36 may be increased or decreased respectively. In other configurations, in addition to or in substitution with the anti-infection drug 42, an immunosuppressant drug (not shown) may be coated on or impregnated within the plurality of fixations projections 36 and/or the first material 34. The immunosuppressant drug may prevent the body's natural rejection mechanisms from attacking the implantable components. The distal end 40 of the plurality of fixations projections 36 may further be atraumatic in shape, for example, rounded as to not harm surrounding tissue. In other configurations, the distal end 40 may define a hook, barb, or other like shape to attach to the surrounding tissue to mitigate migration of the implantable component.

Referring now to FIG. 5, attached to the fixation device 32 may be a one or more anchors 44 configured to engage, for example, at least a portion of the driveline 26. In one configuration a first anchor 46 is disposed at a proximal end of the fixation device 32 and a second anchor 48 is disposed at a distal end of the fixation device. The anchors 44 may be configured to contour the driveline 26, whether the tubular body 28 or the fabric 30, and to sandwich the fixation device 32 between the driveline 26 and the anchors 44 prevent migration of the fixation device 32. The anchors 44 may be flexible such that that may be tightened by, for example, a suture 50 wrapped around at least a portion of the anchor to secure to the anchors 44 to the driveline 26. In one configuration, the first anchor 46 and the second anchor 48 may be sleeves that are slideable over the fixation device 32 to retain the fixation device 32 in place. In other configurations, the anchors 44 are prefabricated with the fixation device 32 such that they are affixed together. In another configuration, the fixation device 32 may be wrapped around the exterior of the plurality of anchors 44. In one configuration, to promote attachment the implantable component, the fixation device 32 may include the plurality of fixations projections 36 on both sides of the first material 34, such that, for example, the plurality of fixations projections 36 are in contact with the fabric 30 and the surrounding tissue. In such a configuration, the fixation device 32 may be wrapped about itself to improve fixation about the fabric 30 or tubular body 28. In other configurations, the plurality of fixations projections 36 are disposed on a single side of the first material 34.

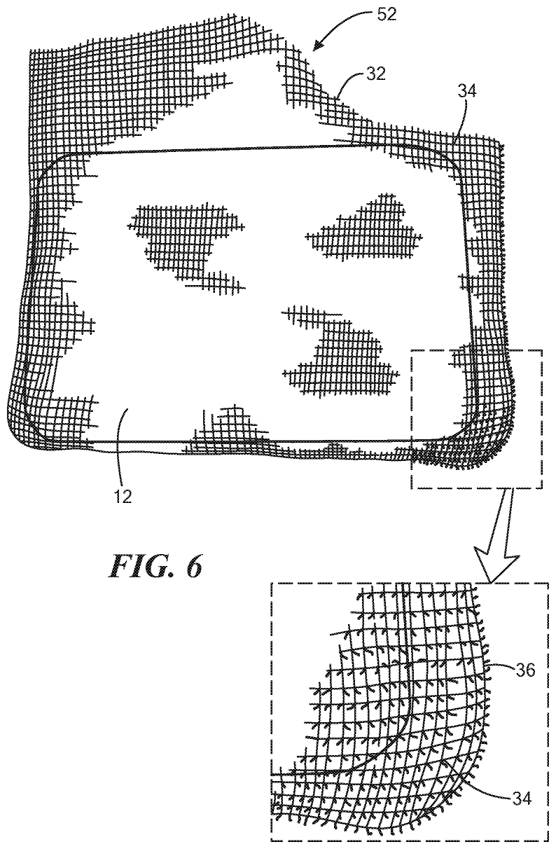

Referring now to FIG. 6, in one configuration, the fixation device 32 may define an enclosure 52, such as a pouch, configured to retain one or more implantable components. For example, the controller 12 may be disposed within the enclosure 52 such that the fixations projections 36 extend outward away from the controller 12. In the configuration shown in FIG. 6, the controller 12 is disposed within the pouch such that every surface of the controller is covered by the fixation devices. In other configurations, the fixation device 32 may be sewn, glued, or otherwise coupled to at least a portion of the controller 12.

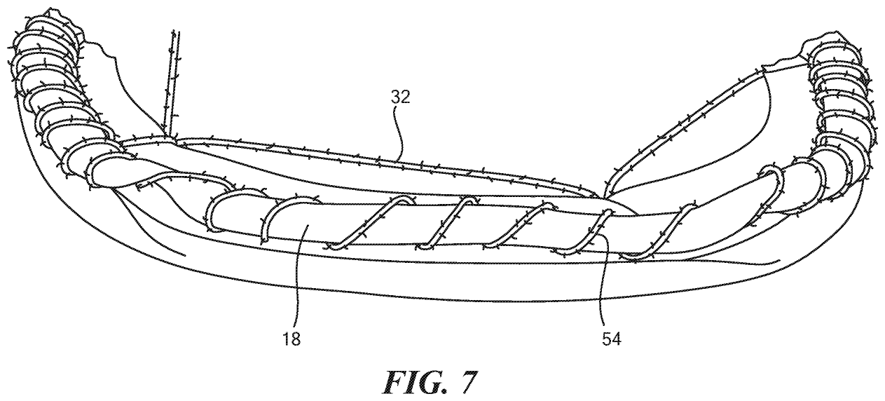

Referring now to FIG. 7, in another configuration, the fixation device 32 may define a filament 54 that wraps around at least a portion of the implantable component. For example, as shown in FIG. 7, the fixation device 32 defines a filament 54, which may include the first material 34 and the fixations projections 36 to provide for fixation. In the example shown in FIG. 7, the fixation device 32 is wound about a portion of a TETS receiver 18, in particular, a perimeter of the TETS receiver 18, but may be wound about any of the implantable components described herein.

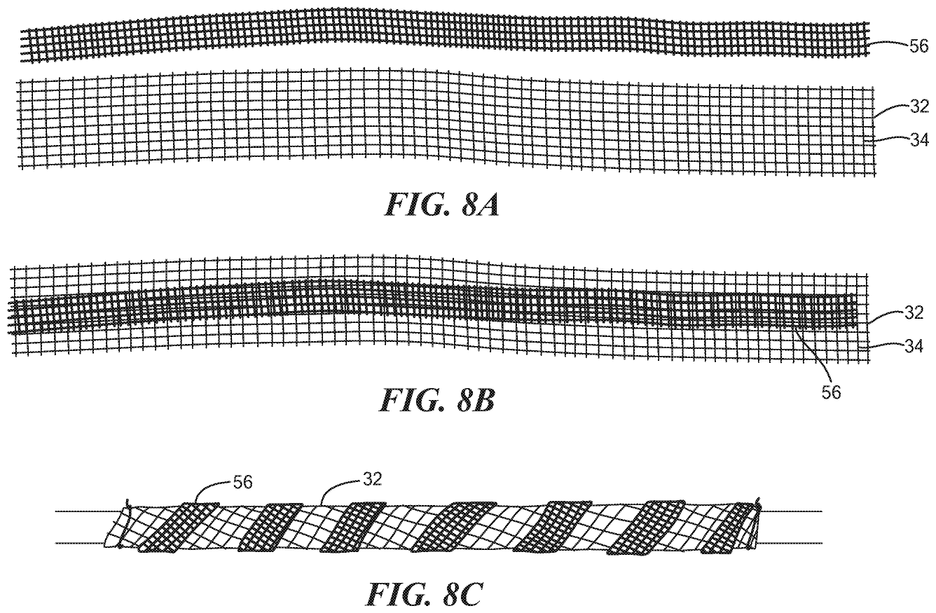

Referring now to FIGS. 8A-8C, in another configuration, the anti-infection drug or coating 42 may be impregnated within or otherwise coated on a bioresorbable or non-bioresorbable mesh 56, for example, TYRX®, sized and configured to be disposed on the first material 34 of the fixation device 32. The plurality of fixation projections 36 may grip and/or interlock within and retain the mesh 56. In other configurations, the mesh 56 is not coated with an anti-infection drug. The mesh 56 may define any shape, size, or pattern on the first material 34. In other configurations, the first material 34 does not include the plurality of fixation projections 36 and the mesh 56 is overlaid on the first material 34. In one configuration, the mesh 56 along with first material 34 is spiral wrapped about the fabric 30 or directly onto the driveline 26. For example, as shown in in FIG. 8C, the mesh 56 is disposed on the surface of the first material 34 and wrapped about the fabric 30. In one configuration, each spiral wrapped section covers and overlaps about 0-100% of the previous section to provide for inter-locking of the first material 34 onto itself. The angle of the overlap may be between 30 to 60 degrees. In an exemplary configuration, the overlap is between 10-30% and an angle for 45 degrees. In other configurations, the mesh 56 is directly spiral wrapped about the fabric 30 without the first material 34 in the manner described above. The spiral wrap may further provide a fish scale type effect that increases stability/pull force acutely.

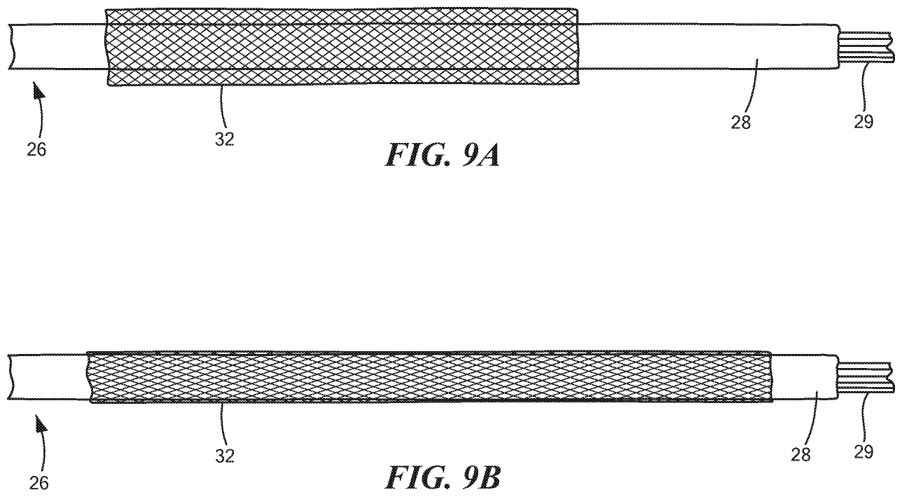

Referring now to FIGS. 9A and 9B, the fixation device 32 may define a tube and may further include the anti-infection mesh 56 disposed about the fixation device 32. In one configuration, as shown in FIG. 9A, the fixation device 32 defines a circumference greater than the of the tubular body 28 such that when it is stretched, as shown in FIG. 9B, it contours the tubular body. Anchors 44 may also be included in a similar manner as described above, or alternatively, the fixation device 32 may be sewn, glued, or otherwise attached to the tubular body 28 or fabric 30. In one configuration, two pairs of silicone rings (not shown) may be disposed about the tubular body 28 or fabric 30 to create corresponding grooves between each corresponding pair of rings. In such a configuration, the fixation device 32 may be sewn into each corresponding groove.

Although described herein with respect to TETS components and related medical implants, it is contemplated that the fixation device 32 and or/mesh 56 may be used with any medical implant in any part of the body.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical implant fixation device, comprising:
at least one from the group consisting of a non-bioresorbable material and a bioresorbable material sized and configured to one from the group consisting of receive and surround the medical implant and be affixed to a surface of the medical implant;
a plurality of at least one from the group consisting of bioresorbable and non-bioresorbable fixation projections extending from the at least one from the group consisting of a non-bioresorbable material and a bioresorbable material; and
at least one from the group consisting of the plurality of bioresorbable and non-bioresorbable fixation projections and the at least one from the group consisting of a non-bioresorbable material and a bioresorbable material sized being coated with at least one anti-infection coating.

2. The device of claim 1, wherein the medical implant is a controller for a ventricular assist device, and wherein the material is non-bioresorbable, and wherein the non-bioresorbable material further defines a pouch sized to receive and enclose the controller.

3. The device of claim 1, wherein the medical implant is a driveline for a ventricular assist device, and wherein the material is non-bioresorbable, and wherein the non-bioresorbable material defines a mesh is configured to be wrapped around and adhered to the driveline.

4. The device of claim 3, wherein the driveline includes an elongated tubular body having at least one electrical connector configured to electrically connect with at least a portion of the ventricular assist device, and wherein the driveline further includes a biocompatible fabric wrapped around at least a portion of the elongated tubular body configured to promote tissue ingrowth, and wherein the non-bioresorbable material is wrapped around the biocompatible fabric.

5. The device of claim 3, wherein the driveline includes a plurality of anchoring sleeves disposed about the driveline, and wherein non-bioresorbable material is disposed between two of the plurality of anchoring sleeves, and wherein the anchoring sleeves are configured to retain the non-bioresorbable material about the driveline.

6. The device of claim 1, wherein the material is non-bioresorbable, and wherein the non-bioresorbable material defines a first side and a second side opposite the first side, and wherein the plurality of bioresorbable fixation projections is disposed on both the first side and the second side.

7. The device of claim 1, wherein the medical implant is a transcutaneous energy coil.

8. The device of claim 7, wherein the material is non-bioresorbable, and wherein the non-bioresorbable material is affixed around a perimeter of the transcutaneous energy coil.

9. The device of claim 8, wherein the non-bioresorbable material is configured to be substantially planar with an outer surface of the transcutaneous energy coil.

10. The device of claim 1, wherein the plurality of bioresorbable fixation projections define one from the group consisting of a plurality of barbs and a plurality of hooks.

11. A method of implanting a medical implant within a patient, comprising:
one from the group consisting of enclosing the medical implant within a fixation device and affixing the fixation device to the medical implant, the fixation device including:
a non-bioresorbable material;
a plurality of bioresorbable fixation projections extending from the non-bioresorbable material; and
at least one from the group consisting of the plurality of bioresorbable fixation projections and the non-bioresorbable material being coated with at least one anti-infection drug; and
implanting the medical implant and the fixation device within the patient.

12. The method of claim 11, wherein the medical implant is a controller for a ventricular assist device, and wherein the controller is enclosed within the fixation device.

13. The method of claim 11, wherein the medical implant is a driveline for a ventricular assist device, and wherein the fixation device is affixed to at least a portion of the driveline.

14. The method of claim 13, wherein the fixation device is wrapped around at least portion of the driveline.

15. The method of claim 14, wherein the driveline includes an elongated tubular body having at least one electrical connector configured to electrically connect with at least a portion of the ventricular assist device, and wherein the driveline further includes a biocompatible fabric wrapped around at least a portion of the elongated tubular body configured to promote tissue ingrowth, and wherein the non-bioresorbable material is wrapped around the biocompatible fabric.

16. The method of claim 11, wherein the non-bioresorbable material defines a first side and a second side opposite the first side, and wherein the plurality of bioresorbable fixation projections is disposed on both the first side and the second side.

17. The method of claim 11, wherein the medical implant is a transcutaneous energy coil.

18. The method of claim 17, wherein the non-bioresorbable material is affixed around a perimeter of the transcutaneous energy coil.

19. The method of claim 18, wherein the non-bioresorbable material is sewn onto the transcutaneous energy coil.

20. A medical implant fixation device, comprising:
a non-bioresorbable material sized and configured to receive and enclose a controller for a ventricular assist device, an entirety of the non-bioresorbable material defining a mesh having a surface area;
a plurality of bioresorbable fixation projections extending from the non-bioresorbable material, the plurality of bioresorbable fixation projections being disposed on substantially the entirety of the surface area, the plurality of bioresorbable fixation projections being configured to engage and anchor to subcutaneous adipose tissue; and
the plurality of bioresorbable fixation projections being coated with at least one anti-bacterial drug.

21. A medical implant fixation device, comprising:
at least one from the group consisting of a non-bioresorbable material and a bioresorbable material sized and configured to one from the group consisting of receive and surround the medical implant and be affixed to a surface of the medical implant;
a plurality of bioresorbable fixation projections extending from the at least one from the group consisting of a non-bioresorbable material and a bioresorbable material; and
a mesh being disposed on the at least one from the group consisting of the non-bioresorbable material and the bioresorbable material.

22. The device of claim 21, wherein the mesh includes at least one from the group consisting of anti-infection drug and a silver-ion coating on its surface.

23. The device of claim 22, wherein the mesh is wrapped about the at least one from the group consisting of the non-bioresorbable material and the bioresorbable material.
